(19) United States
(12) Patent Application Publication
(10) Pub. No.: US 2016/0015285 A1
(43) Pub. Date: Jan. 21, 2016

(54) OPTOELECTRONIC REMOTELY POWERED SILICON BASED HYBRID NEURAL ELECTRODE

(71) Applicant: Drexel University, Philadelphia, PA (US)

(72) Inventor: Karen Moxon, Collingswood, NJ (US)

(21) Appl. No.: 14/801,910

(22) Filed: Jul. 17, 2015

Related U.S. Application Data
(60) Provisional application No. 62/025,745, filed on Jul. 17, 2014.

(51) Int. Cl. A61B 5/04; A61B 5/00

(52) U.S. Cl. CPC A61B 5/04001; A61B 5/686

(57) ABSTRACT

One aspect of the invention provides an optically powered, integrated wireless neural electrode-telemetry module comprising (a) porous silicon wafer; (b) at least one neural electrode; (c) a low noise pre-amplifier; (d) an optical power converter; (e) a signal processor; and (f) a radio-frequency (RF) transmitter microchip. Another aspect of the invention provides a method of chronically recording electrical activity from a single neuron in vivo. The method comprises implanting an optically powered, integrated wireless neural electrode-telemetry module into the brain or spinal cord of a subject.

### CROSS-REFERENCE TO RELATED APPLICATION

This application claims priority under 35 U.S.C. §119(e) to U.S. Provisional Patent Application Ser. No. 62/025,745, filed Jul. 17, 2014. The entire content of this application is hereby incorporated by reference herein.

### STATEMENT REGARDING FEDERLLY SPONSORED RESEARCH OR DEVELOPMENT

The invention was made, in part, using funds obtained under National Institute of Health SBIR Grant No. 5R43NS046957-02. The U.S. Government has certain rights in this invention.

### BACKGROUND OF THE INVENTION

The possibility for interfacing the nervous system with electronic devices has long fascinated scientists, engineers, and physicians. In general, an ability to expand the bandwidth of communication between brain and machine would provide many interesting possibilities, ranging from faster human-computer interfaces to direct remote control (i.e., “telekinesis”). In medicine, the field of neuroprosthetics has grown rapidly to include a variety of devices for stimulating peripheral nerve tissue, several of which are now available commercially.

However, devices that interact with brain tissue have lagged behind due to the sensitivity of brain tissue to the introduction of the electrode.

### SUMMARY OF THE INVENTION

In one embodiment, the present invention provides an optically powered, integrated wireless neural electrode-telemetry module. The electrode-telemetry module comprises (a) a porous silicon wafer; (b) at least one neural electrode; (c) a low noise pre-amplifier; (d) an optical power converter; (e) a signal processor; and (f) a radio-frequency (RF) transmitter microchip.

In one embodiment, the optical power converter is adapted and configured to receive power from red or near-infrared illumination transmitted through a skull. In another embodiment, the optical power converter is adapted and configured to receive power from optical signals carried by a fiber optic cable, wherein the fiber optic cable terminates near the optical power converter without physically contacting the optical power converter.

In one embodiment, the porous silicon wafer comprises at least one pharmacological agent. The pharmacological agent may be brain derived neural growth factors (BDNF), nerve growth factor (NGF), Poloxamer 188, a cell adhesion molecule, glial filament associated proteins (GFAP), intermediate filament associated proteins (IFAP), endothelin-1, cytokinen, IL-6, and any combination thereof.

In another aspect, the present invention provides a method of chronically recording electrical activity from a single neuron in vivo. The method comprises the steps of (a) implanting an optically powered, integrated wireless neural electrode-telemetry module into the brain or spinal cord of a subject, wherein the module comprises a porous silicon wafer; at least one neural electrode; a low noise pre-amplifier; an optical power converter; a signal processor; and a radio-frequency (RF) transmitter microchip; (b) providing an optical signal to power the optical power converter; and (c) receiving modulated radio signals from the RF transmitter microchip, wherein the modulated radio signals correspond to electrical activity of a single neuron.

In one embodiment, the optical signal comprises red or near infrared illumination transmitted through a skull. In another embodiment, the optical signal is carried by a fiber optic cable, wherein the fiber optic cable terminates near the optical power converter without physically contacting the optical power converter. In yet another embodiment, the porous silicon wafer comprises a pharmacological agent. The pharmacological agent may be brain derived neural growth factors (BDNF), nerve growth factor (NGF), Poloxamer 188, a cell adhesion molecule, glial filament associated proteins (GFAP), intermediate filament associated proteins (IFAP), endothelin-1, cytokinen, IL-6, and any combination thereof.

In another embodiment, the subject is an animal. The animal may be a mammal. The mammal may be a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic illustration of a proposed wireless multi-channel neural electrode module monolithically-integrated with its own laser-power converter, signal processor, and radio frequency transmitter.

FIGS. 2A and 2B are a series of graphs depicting the significant transparency of both the human skull and soft-tissue using red-to-infrared (IR) illumination. FIG. 2A is a graph depicting the wavelength dependence of the absorption coefficient of human cranial bone in vitro. FIG. 2B is a graph depicting estimated tissue penetration as a function of wavelength for uncolored tissue, defined as 1/(ₐ+ₛ).

FIGS. 3A-3F are a series of images depicting schematic illustrations of typical porous Silicon (Si) morphologies. FIG. 3A is an image depicting an n (100)-oriented Si wafer anodized in the dark. FIG. 3B is an image depicting an anodized n (110)-oriented Si wafer. FIG. 3C is an image depicting an n (100)-oriented Si wafer anodized with backside illumination. FIG. 3D is an image depicting a p− wafer anodized with dilute ethanoic HF electrolyte. FIG. 3E is an image depicting an n+ Si wafer anodized with dilute aqueous HF electrolyte. FIG. 3F is an image depicting a p− Si wafer with concentrated aqueous HF.

FIG. 4 is a series of images depicting representative images of several types of porous silicon structures produced. The leftmost panel is an image depicting a cross-sectional view of porous silicon. The four rightmost panels are images depicting a top view of several types of porous silicon.

FIG. 5 is a series of images depicting a silicon electrode and representative recordings of electrical activity obtained from the electrode. The leftmost panel is an image of a silicon-based microelectrode comprising four (4) neuron electrodes. The scale bar is 1 mm (1,000 microns). The rightmost panel is a series of traces depicting extracellular electrical activity recorded using an electrode such as the one shown in the leftmost panel. The horizontal scale bar represents 100 ms and the vertical scale bar represents 100 V.

FIGS. 6A-6C are a series of plots depicting representative recordings and analyses of recordings of electrical activity of interneurons obtained from electrodes chronically implanted in the mesial temporal lobe (MTL). FIGS. 6A-6C are graphs illustrating interneuron activity and interneuron field coherence prior to and after seizure onset in the MTL. In FIGS. 6A-6C, a time scale change occurs at 10 minutes prior to seizure. This is denoted in each graph by the solid vertical line on the left. FIG. 6A depicts a peri-seizure time histogram of interneuron activity aligned to seizure onset in the MTL (solid vertical line, right) and averaged over all seizures across all patients. The solid horizontal line indicates mean firing rate for a 30 minute baseline period occurring 1 hour prior to electrographic seizure onset in the MTL. Dashed horizontal lines indicate +/− 3 standard deviations from baseline firing activity. Below the histogram, lines indicate a decimated rastergram of example neurons showing reduced firing rate prior to seizure. In FIGS. 6B-6C, the dashed vertical line indicates a 20 second pre-ictal window of interest where interneuron firing rate significantly decreased compared to baseline activity. There appears to be a trend towards decreasing interneuron activity in the 5 minutes prior to seizure, but this drop only reaches significance 20 seconds prior to seizure onset in MTL. Within this window, 91% of interneurons exhibited a drop in firing rate compared to baseline activity. FIG. 6B depicts an average normalized power spectrum showing no pre-ictal changes and a shift towards ictal frequencies after seizure onset. FIG. 6C depicts averaged interneuron-field coherence showing a significant elevation in coherence in the 20 seconds prior to seizure onset in MTL. This activity occurs predominantly below 11 Hz and coincides with the window of time where seizure activity has started in the neocortex but has not yet reached the MTL.

FIGS. 7A-7B are a series of graphs depicting representative recordings and analyses of recordings of electrical activity of pyramidal cells obtained from electrodes chronically implanted in the mesial temporal lobe (MTL). FIGS. 7A-7B illustrate pyramidal cell activity and pyramidal cell field coherence prior to and after seizure onset in the MTL. In FIGS. 7A-7B, a time scale change occurs at 10 minutes prior to seizure. This is denoted in each graph by the solid vertical line on the left. FIG. 7A depicts a peri-seizure time histogram of pyramidal cell activity aligned to seizure onset in the MTL (solid vertical line, right) and averaged over all seizures across all patients. The solid horizontal line indicates mean firing rate for a 30 minute baseline period occurring 1 hour prior to electrographic seizure onset in the MTL. The dashed horizontal lines indicate +/− 3 standard deviations from baseline firing activity. Below the histogram, lines indicate a decimated rastergram of several individual neurons. No significant changes in firing rate occurred in the 5 minutes prior to seizure onset in MTL. FIG. 5B depicts averaged pyramidal cell-field coherence showing no significant change in coherence in the 20 seconds prior to seizure onset in MTL as compared to baseline, unlike interneuron-field coherence. The dashed vertical line indicates a 20-second pre-ictal window of interest where interneuron firing rate decreased significantly compared to baseline activity.

FIGS. 8A-8B are graphs illustrating information flow from seizure focus to MTL structures, measured by chronically implanted electrodes. FIG. 8A depicts directed field-field coherence spectrograms which show an increase in information flow from seizure focus to downstream MTL structures in the 20 seconds prior to electrographic seizure onset in the MTL. At the same time, no significant reverse information flow was observed suggesting that seizure propagation form the neocortex is associated with a unidirectional flow of ictal information. FIG. 8B depicts examples of simultaneous local field potential recordings in the seizure focus (left) and the MTL (right) that share dominant oscillation frequencies. Vertical scale bars represent 1 mV while horizontal scale bars represent 1 second.

FIGS. 9A-9C are graphs illustrating an assessment of recruitment of MTL into seizure. FIG. 9A and FIG. 9B illustrate example local field potentials of seizure event with multiunit rastergram. The vertical scale bar represents 1 mV. Lettered subplots show expanded 3 second windows of activity corresponding to horizontal bars. Both seizure events exhibit an increase in rhythmic ictal spiking and concomitant rhythmic multiunit activity as the seizure event progresses. In FIG. 9C, the top graph depicts a two second window of large amplitude ictal spiking. The middle of FIG. 9C depicts an accompanying wavelet power scalogram (0-3 kHz) and the bottom of FIG. 9C shows an expanded view of high frequency wavelet power above 400 Hz. Distinct islands of increased high frequency power are observable consistent with multiunit activity rather than the stereotypic tapered cone pattern seen with filtering artifact.

### DETAILED DESCRIPTION OF THE INVENTION

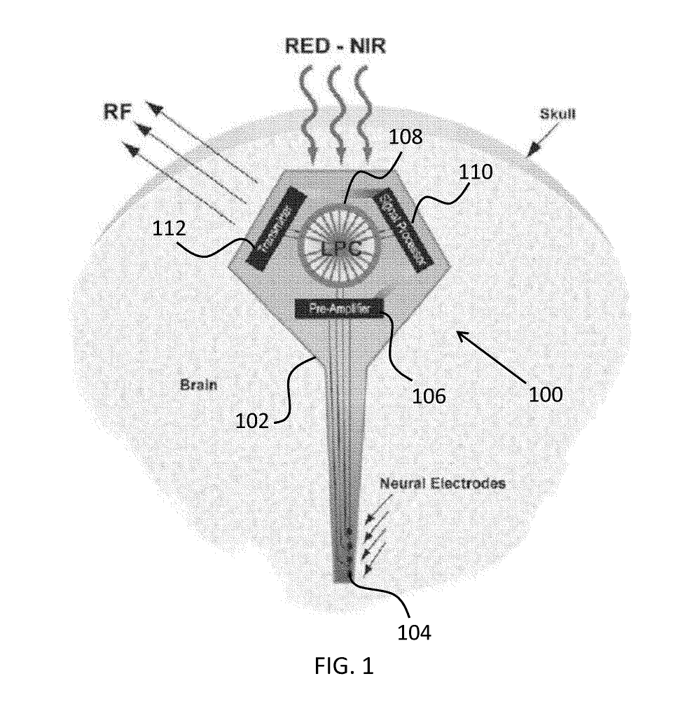

Referring to FIG. 1, one embodiment of the present invention provides a wireless, optically-powered neural electrode device 100 including a porous silicon substrate 102, at least one neural electrode 104, a low-noise, high-gain pre-amplifier 106, an optical power converter 108, a signal processor 110, and a radio-frequency (RF) transmitter microchip 112. The neural electrode device 100 can be powered by an optical energy source 114. The purpose of the present invention is to provide a device for long-term chronic single unit (one neuron) recordings from at least one mammalian neuron by implanting the device in the brain or spinal column of an animal.

The present invention provides several key inventive steps over other chronic electrodes. First, the device of the present invention eliminates the need for the device to be physically tethered to either a power supply or a transceiver for sending data. By removing this physical tether, the present device overcomes a significant source of immunological reaction by the host animal that receives the implant, thereby reducing glial scarring and prolonging the functional lifetime of the device. Second, the device of the present invention provides for unique porous silicon formulations that enhance the bonding, adsorption, adherence, and long-term delivery of various pharmacological and therapeutic agents that are useful in preventing an immune response to the device and subsequent glial scarring as well as providing support for neural growth and maintenance.

### Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent on the context in which it is used.

As used in the specification and claims, the terms “comprises,” “comprising,” “containing,” “having,” and the like can have the meaning ascribed to them in U.S. patent law and can mean “includes,” “including,” and the like. Unless specifically stated or obvious from context, the term “or,” as used herein, is understood to be inclusive.

The term “epilepsy” as used herein means a neurological disorder characterized by repeated seizures.

The term “seizure” as used herein means a transient symptom of abnormal, excessive or synchronous neuronal activity in the brain.

A “subject” shall be understood to include any mammal including, but not limited to, humans. The term “subject” specifically includes rats. The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Silicon” or “Si,” as used herein, refers to the most common semiconductor. The term “doping” as used herein, refers to the process of introducing an atom or another element into the silicon crystal to alter its electrical properties. The dopant has either three or five valence electrons, as opposed to silicon's four. Phosphorus atoms, which have five valence electrons, are used for doping n-type silicon (because phosphorous provides its fifth, free, electron). A phosphorus atom occupies the same place in the crystal lattice that was occupied formerly by the silicon atom it replaced. Four of its valence electrons take over the bonding responsibilities of the four silicon valence electrons that they replaced. But the fifth valence electron remains free, without bonding responsibilities. When numerous phosphorus atoms are substituted for silicon in a crystal, many free electrons become available. Substituting a phosphorus atom (with five valence electrons) for a silicon atom in a silicon crystal leaves an extra, unbonded electron that is relatively free to move around the crystal. The most common method of doping is to coat the top of a layer of silicon with phosphorus and then heat the surface. This allows the phosphorus atoms to diffuse into the silicon. The temperature is then lowered so that the rate of diffusion drops to zero. Other methods of introducing phosphorus into silicon include gaseous diffusion, a liquid dopant spray-on process, and a technique in which phosphorus ions are driven precisely into the surf ace of the silicon. Boron, which has three valence electrons, is used for doping p-type silicon. When a boron atom assumes a position in the crystal lattice formerly occupied by a silicon atom, there is a bond missing an electron (in other words, an extra hole). Substituting a boron atom (with three valence electrons) for a silicon atom in a silicon crystal leaves a hole (a bond missing an electron) that is relatively free to move around the crystal. P-type doping creates an abundance of holes, or free positive charge carriers.

The term “N-Type silicon” as used herein refers to a property of the silicon semiconductor material. N-type silicon may be doped with either Arsenic (As), antimony (Sb), or phosphorous (P). Doping with donor atoms increases the density of negative charge carriers (electrons) in the conduction band and produces an N-type semiconductor.

The term “P-type silicon” as used herein, refers to silicon doped with Boron (B) or gallium. P-type doping creates an abundance of holes. Boron and gallium each have only three outer electrons. When mixed into the silicon lattice, they form “holes” in the lattice where a silicon electron has nothing to bond to. The absence of an electron creates the effect of a positive charge, hence the name P-type. Holes can conduct current by accepting an electron from a neighbor.

Abbreviations of different types of silicon useful in the present invention are summarized in Table 1 below.

TABLE 1

| | |
|---|---|
| N | Silicon doped to create excess negative charge carriers (electrons) |
| N+ | Heavily doped, N-type silicon |
| P | Silicon doped to create excess positive charge carriers (holes) |
| P− | Lightly doped P-type silicon wafer |
| P+ | Heavily doped P-type silicon wafer |
| PIP+ | Lightly doped P-type epilayer on a heavily doped P-type substrate |
| PIP− | Lightly doped P-type epilayer on a lightly doped P-type substrate |
| P− | Typically P-type material with a resistance greater than 1 cm |
| P+ | Typically P-type material with a resistance less than 1 cm |
| P++ | P-type material highly doped with boron with a resistivity between |
| | 0.005 cm and 0.010 cm |

Within this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6. This applies regardless of the breadth of the range.

### Description

The present invention is based on the discovery of devices and methods that allow long-term, chronic, in vivo recording from a single neuron when the device is implanted in the brain or spinal cord of a subject.

Referring again to FIG. 1, one embodiment of the present invention provides an optically powered, wireless neural electrode-telemetry module 100 including: a porous silicon substrate 102, at least one neural electrode 104, a low noise pre-amplifier 106, an optical power converter 108, a signal processor 110, and a radio-frequency (RF) transmitter microchip 112.

The porous silicon substrate 102 is preferably less than or equal to 50 microns in thickness, having a narrow tip at one end ranging up to 0.1 mm in width and a wider region at the opposite end approximately twice the width of the narrow tip. The wider region contains bonding pads and is sized to be large enough to house and provide contacts for the integrated electronics 106, 108, 110, 112.

One function of the porous silicon substrate 102 is as a scaffold to promote neural repair and growth after implantation. The porous silicon wafer 102 of the invention is produced so as to have deep pores that can be impregnated or coated with a pharmacological agent that promotes the growth of neurites into the porous silicon, reduces inflammation, or prevents glial scarring. A pharmacological agent of the present invention comprises a drug, compound, molecule, or other agent that promotes the growth of neurons and neuritis, reduces inflammation, or reduces glial scarring.

Examples of such agents include, but are not limited to, neurotrophic agents, such as brain derived neural growth factors (BDNF), nerve growth factor (NGF), Poloxamer 188 and the like, cell adhesion molecules, and any other compound or molecules that support, maintain, or promote the growth of neurons and neuritis. Other compounds or agents useful in the practice of the invention include glial filament associated proteins (GFAP), intermediate filament associated proteins (IFAP), endothelin-1, and cytokinen, IL-6. The present invention should not be considered to be limited to the compounds recited herein but should be considered to encompass any compound, drug, molecule or agent, known or unknown, which is able to support, maintain, or promote the growth of a neuron as well as inhibit the formation of a glial scar. It will be appreciated by one skilled in the art that a single porous silicon substrate may comprise more than one pharmacological agent. Another function of the porous silicon substrate is as a scaffold to support the integrated electronics of the neural electrode-telemetry module.

The present invention encompasses optimizing the morphology of porous silicon to promote neurite growth as well as adsorption, coating, or binding of a pharmacological agent to the porous silicon substrate. The silicon may be n-type or p-type, or a combination thereof, depending on the dopants used in the manufacture of the silicon wafer. Miniaturized electronic components 104, 106, 108, 110, 112 of the neural electrode-telemetry module are patterned onto the silicon substrate. The device 100 can include at least one neural electrode 104, a low noise pre-amplifier 106, an optical power converter 108, a signal processor 110, and a radio-frequency (RF) transmitter microchip 112.

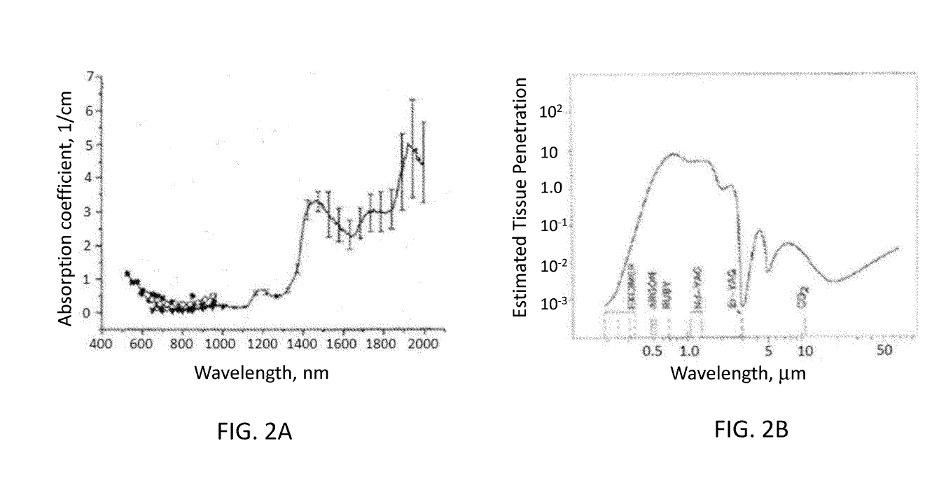

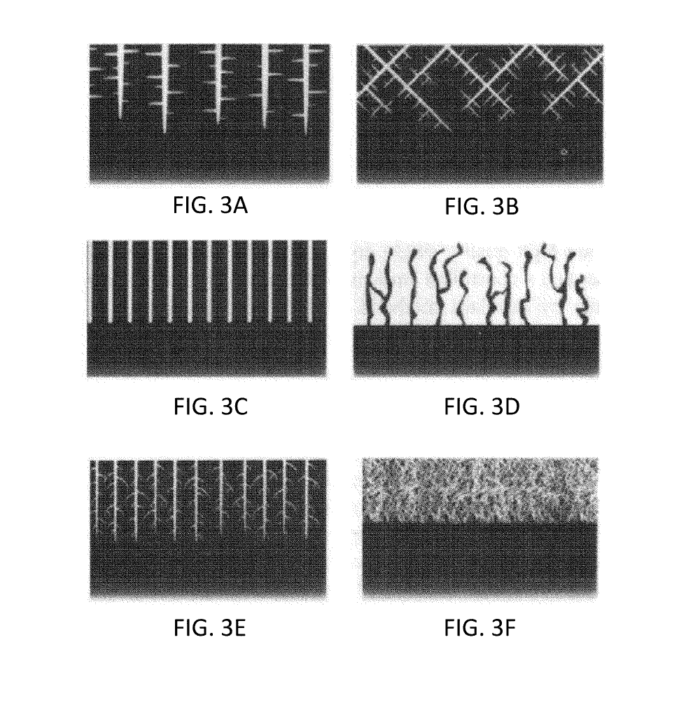

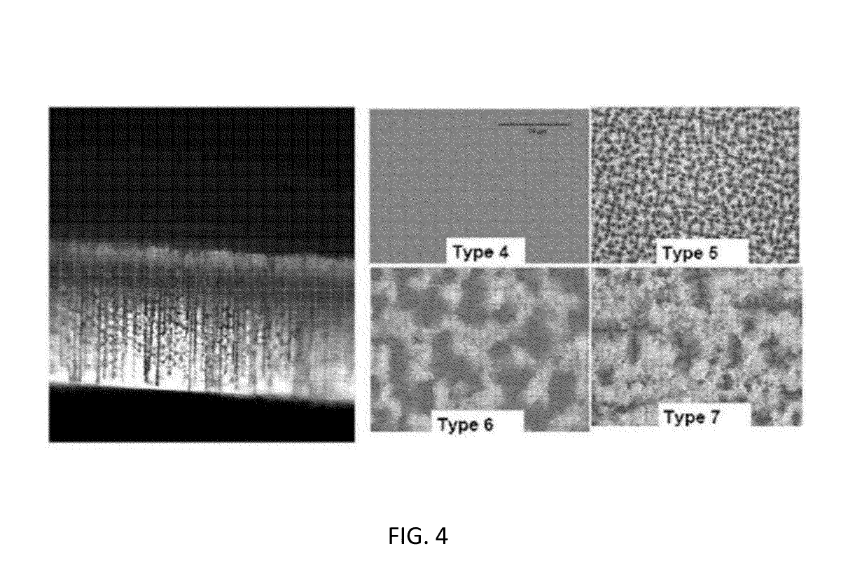

The optical power converter 108 provides electrical power to drive a low-noise, high-gain pre-amplifier 106, a signal processor 110, and a radio frequency transmitter chip 112. The device 100 of the present invention need not be physically attached to a power supply or other electronics, but rather is remotely powered and sends its signals via a radio frequency transmitter chip 112. This arrangement is a significant advance over currently available chronically implanted electrodes because it eliminates a significant source of immune response and glial scarring. In one embodiment, the optical power converter 108 is a laser-power converter powered by red to near-infrared (NIR) illumination through the skull (FIGS. 2A and 2B). In another embodiment, the optical power converter 108 receives optical input via a fiber optic cable positioned close to, but not attached to, the optically powered neural electrode-telemetry module 100. Exemplary optical power sources include excimer lasers, ultraviolet lasers, ruby lasers, neodymium-doped yttrium aluminium garnet (Nd:YAG) lasers, CO₂ lasers, and the like. The porous silicon substrate 102 is patterned with multiple recording sites 104, preferably 2 to 32 recording sites, at the narrow tip of the substrate 102. Each recording site 104 is connected to a bonding pad placed at the opposite wider end of the substrate 102 via a conducting line that runs from the recording site 104 to the bonding pad. A metal layer, preferably platinum, is applied to the entire pattern to produce the recording sites 104, conducting lines and bonding pads which form the electrode. A first insulating layer, preferably alumina, is then applied to the metal conducting lines and bonding pads. In a preferred embodiment, the alumina is ionized using a process of ion-beam assisted deposition.

Application of a second insulating layer comprising polyimide to the conducting lines is also preferred. To complete the array, the bonding pads are attached to second stage recording equipment which amplifies and filters the signal. Examples of second stage recording equipment include, but are not limited to, the MNAP system available from Plexon Inc. of Dallas, Tex. the Experimenters Workbench system available from DataWave Technologies of Loveland, Colo. and Alpha-Map devices from Alpha Omega Co. USA Inc. of Alpharetta, Ga.

The neural electrode-telemetry module of present invention are preferably sized to mimic single microwires. However, unlike single microwires, the multiple recording sites of the CBMSE arrays can be integrated with onboard electronics in a VLSI circuit to permit large numbers of neurons to be measured simultaneously as described in Karen A. Moxon et al., “Designing a Brain-Machine Interface for Neuroprosthetic Control in Neural Prosthesis for Restoration of Sensory and Motor Function” in Neural Prostheses for Restoration of Sensory and Motor Function (John K. Chapin & Karen A. Moxon eds. 2000).

Electrical signals emanating from the neural electrodes 104, expected to be from about 1 V to about 10 V, are amplified by the pre-amplifier 106, electronically processed by the signal processor 110 and then modulate the radio signal from the RF transmitter 112. Certain embodiments of the present invention provide for multichannel recording from multiple neurons by implanting a module of the invention that comprises multiple neural electrodes 104. These electrodes 104 can be offset along the vertical- or horizontal- axis of the electrode and allow for simultaneous recordings from multiple neurons. The data from these multiple electrodes 104 can be processed and transmitted simultaneously.

The present invention also provides methods of using a neural electrode-telemetry module to record from a neuron in a subject. One or more of the electrode-telemetry modules 100 can be implanted into the cortex or spinal cord of a subject. If the optical power converter 108 requires that a signal be conducted via a fiber optic cable, the fiber optic cable can be positioned such that optical signals are transmitted to the optical power converter 108. During the process of implantation, the position of the electrode-telemetry module and any associated fiber optic cable can be adjusted to optimize recordings from single neurons.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The promise of BMI for restoration of sensory and motor function in patients with neurological disease or injury has been hindered by an inability to record single neuron activity from the brain for long periods of time. The general problem is that microelectrodes can only record single neurons for approximately 3-12 months (in rats and monkeys, respectively) before the electrode fails.

This is especially important when patients that are already physically compromised undergo an invasive surgery to have electrodes implanted and the electrodes fail. One reason for the failure is the response of the brain toward the implanted microelectrode that produces a glial scar between the electrode and healthy neurons preventing further recordings. The ability to interface with the human central nervous system (CNS) for therapeutic relief, to replace injured communication lines or to add additional communication lines to increase the bandwidth between the CNS and the environment requires devices that can remain biocompatible in the CNS environment for decades.

### Example 1

### Formation of Nano-Structured Porous Silicon Surfaces that Enhance Neurite Growth into Nanostructures Porous Silicon Structures to Stabilize the Microelectrode and Eliminate the Foreign Body Response

The nanostructured surfaces of the present invention are designed on porous structures that can act as a scaffold. The nanostructured porous surfaces are optimized so that they maintain their bioactive properties and facilitate increased neurite outgrowth and reduced glial proliferation.

### Porous Silicon Wafers

Porous silicon (Si) formations can be produced by varying parameters for electrochemical etching of Silicon. Studies have shown that silicon is safe for use in brain tissue, and excellent recordings have been made acutely. However, the signal deteriorated within the first 24 hours and rarely could the signal be recorded 48 hours after implantation. The cause of this quick degradation is not fully understood, however, much is known about the effects of cutting through brain tissue. Electrodes patterned onto ceramic substrates can record single neuron activity for up to three weeks when implanted chronically into the brain of rats. However, the ideal electrode would be able to record continuously for years. The limiting factor on the recording is likely damage to the surrounding neural tissue when the electrode is introduced in to the brain. If this damage can be minimized or eliminated and neural growth at the recording site can be promoted, recordings from single neurons could be maintained indefinitely.

### Repair Agents Reduce Immunological Response to Electrode Insertion and Mechanical Damage

The initial responses of microglia to brain insult are likely beneficial and include production of neurotrophic substances and cell adhesion molecules, which support injured neurons. Activated cells express glia filament associated proteins (GFAP) near the site of injury and into surrounding tissue and intermediate filament associated proteins (IFAP) at sites near tissue damage. Expression of these proteins appears necessary for restorative events to take place. By enhancing these neurotrophic factors at the time of electrode insertion, the initial brain insult can be minimized and scarring prevented. These events allow the microglia to efficiently take up ions that diffuse from the injured cells. If neurons die during the implantation of the electrodes, the microglia will transform into phagocytotic cells and remove the neural debris. However, if the injury is severe, which is likely the case with the introduction of electrodes, the reactive fibrillary of the astrocytes can create a glial scar, effectively producing a physical barrier between the electrode and healthy cells. Although the extent of scarring depends on the severity of the brain damage, a minimal response around affected neurons is always observed after mild, indirect trauma or axotomy. This glial scarring is composed of a dense network of hypertrophic astrocytes with thick interdigitating processes and associated extracellular matrix. This buildup of glial scar can prevent contact of the electrode with healthy tissue. However, it is possible for phagocytotic microglia to remove cellular debris within a few days, detach from the microglial nodules and downregulate their activation markers which instigates the process.

Reengineering the glial response is two-fold process: first, the damage to neural tissue should be minimized during implantation of the electrode; and second, the neuroglia response should be limited. One method for accomplishing this is to employ neurotrophic factors to minimize negative neural reaction to the implantation of the electrode. The present invention uses nanostructured porous surfaces to create a scaffold for neural repair and growth after electrode implantation. This will enhance the interaction between the neural tissue and the electrode and reduce the damage to neural tissue from implantation of the electrode by limiting the neuroglia response. For example, enhancing brain-derived neural growth factors just after injury may support injured neurons to recover. Increasing the amount of endothelin-1 at the time of electrode insertion may elicit a marked vasoconstriction of cut pial arterioles to reduce contact of blood with neural tissue. Addition of the cytokinen and/or IL-6 has been shown to increase the production of nerve growth factor and decrease production of astrocytes that may support damaged neurons immediately after electrode insertion.

### Example 2

### Quantitatively Assessing Ability of Neurites to Grow into Porous Silicon

PC12 pheochromocytoma cells of chick ganglion cells are grown on one-inch square porous silicon surfaces and allowed to grow to confluence. Medium is replaced every 2-3 days. PC12 pheochromocytoma cells are purchased from American Type Culture Collection (CRL1721) and used without further characterization. The cells are cultured in Dulbecco's Modified Eagle Medium supplemented with 5% horse serum (Sigma), 1% penicillin/streptomycin, and 100 ng/ml of nerve-growth factor (NGF) (Sigma). Cells are maintained under standard cell culture conditions (sterile chamber maintained at 37° C. and a humidified environment (5% CO₂ / 95% air) and used at passage numbers <10.

The growth of neurites (extensions that may form axons) into the porous silicon will be determined per PC12. Cells are rinsed with PBS, fixed with formaldehyde and stained with DiI (D-282, Molecular Probes). The porous silicon samples are then diced and the neurites in a cross section are counted. Images of the cells are also obtained using SEM.

The number of neurites will be determined in at least five random fields per substrate. All experiments will be run in triplicate, and repeated three different times. Comparisons will be made between different types of porous silicon and different concentrations of BDNF or NGF. Numerical data are analyzed using standard analysis of variance (ANOVA) techniques.

### Example 3

### Design of Remote-Powered Hybrid Neural Electrode Module

A principal feature of the present invention is elimination of a physical tether connecting a silicon wafer to a power supply and allow single neuron spike times to be telemetrically transmitted to a microprocessor outside the skull.

The remote-powered neural electrode module consists of a hybrid design, utilizing currently available miniature components which include a laser power converter (LPC), a radio frequency (RF) transmitter microchip, a signal processor, and a low-noise preamplifier.

The low noise preamplifier will amplify the electrical signals emanating from the neural electrodes (whose signal levels are expected to be in the 1-10 V range). The LPC will provide the electrical power to operate the preamplifier as well as the signal processor and radio transceiver. The hybrid module can be designed to operate only when optical power is directed towards the implanted LPC, eliminating the need for an implanted battery. The electrical signals coming from the neural electrodes/preamplifier can be electronically processed by the signal processor and can then modulate the radio signal coming from the RF transmitter. In one design the electronics are always on as long as optical power is applied to the LPC. The total electrical power dissipation is expected to be less than 10 mW, and is therefore not expected to result in any deleterious tissue heating.

### Example 4

### Recording Single Units with Remotely Powered Implanted Electrode

The electrical potential recorded with an extracellular electrode inserted into the brain tissue is the sum of electrical activity around the recording site of the electrode. This electrical activity consists of the electrical fluctuations due to changes in membrane potential of neurons near the recording electrode. The changes in potential can be due to synaptic inputs or voltage dependent ion channels including those that contribute to the generation of an action potential. Changes in potential due to synaptic inputs are small, and contribute to the extracellular potential recorded from a microelectrode, but cannot be individually discriminated because their amplitude is too small. The potential change due to an action potential is much larger and can be recorded if the microelectrode is close enough to the cell, usually within 50 microns. If the microelectrode is too far away, the amplitude of the action potential degrades and is corrupted by other potential changes from other cells and can no longer be discriminated from the background activity. In essence, reflection of the action potential in the external environment is carried out by the external membrane resistance, produced by the salt solution of the extracellular environment.

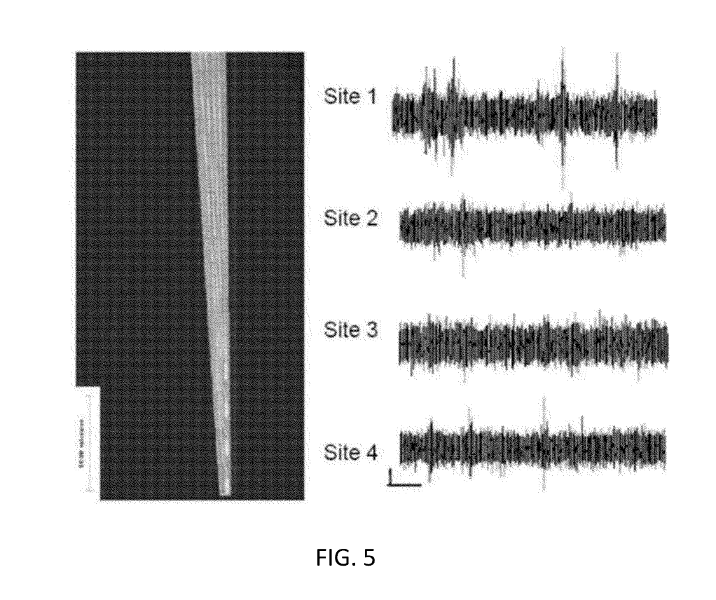

The action potential can generally be recognized or discriminated from the other electrical potentials recorded because of its large amplitude relative to background activity and distinctive shape (FIG. 5). An action potential is caused by the rapid movement of ions across the nerve cell membranes, and is triggered when the influx of positively charged ions reaches a critical value known as the threshold. When this occurs, a brief (1-2 milliseconds) but substantial change in the permeability of the membrane to positively charged ions occurs creating a relatively large (50-100 V) change in the potential recorded by the extracellular electrode if the electrode is close enough to the cell (within about 50 m). These generally look like spikes in the analog signal recorded and the firing of action potentials by neurons are often referred to as spikes.

FIG. 5 shows a photomicrograph of the tip of a first-generation of four-site porous silicon-based microelectrode and tested in vivo using a rat model. The scale bar is 1 mm (1,000 microns). The four recording sites are toward the bottom of the picture. The right panel depicts simultaneous recording of neural activity after the microelectrode was chronically implanted into the brain of a rat. The neural activity represents the continuous analog signal recorded from the extracellular space. Sites 1, 2, and 4 show are obvious spikes in the neural activity representing the action potentials of nearby neurons. At site 3, it is difficult to discriminate spikes from the on-going activity; note, the horizontal scale bar is 100 ms, and the vertical scale bar 100 V. The signal measured from a microelectrode inserted into the extracellular space reflects the voltage change due to this neuronal activity between the electrode tip and a reference electrode inserted somewhere distal to the recording electrode. Because the extracellular space consists of a water-based solution rich in salts, the changes in membrane potential across neurons is readily carried through the extracellular space. The ability of these voltage changes to conduct through the extracellular space are amplitude and frequency dependent.

### Example 5

### Recording Single Neuron Activity with Chronic Electrodes

The present invention provides a device for chronic recording of single neuron activity. The present invention provides for long-term chronic single unit (one neuron) recordings from at least one mammalian neuron by implanting the device in the brain or spinal column of an animal. The device of the present invention can be chronically implanted in any brain region, including deeper brain regions, such as the mesial temporal lobe (MTL).

Single neuron activity recordings from chronically implanted electrodes permit in depth studies of neuronal function. For example, in a study described in U.S. application Ser. No. 14/735,076, recording of single neuron activity from chronically implanted electrodes before and after seizure events enabled investigation of mechanisms underlying seizure propagation to MTL networks.

The device of the present invention can be used to measure patterns of neuronal activity, such as changes in neuron firing rates and neuron synchrony. The device of the present invention can also be used to study various inputs that neurons receive. Inputs received by neurons are the accumulated post-synaptic potentials that arrive from adjacent neurons. The local field potential (LFP) is effectively a summation of this cumulative activity and, so, to understand changes that occur in firing rate (neuronal output), the firing rate can be related to the local field potential (neuronal input). To measure neuronal synchrony between firing rates and local field potentials, a measure called the unit-field coherence can be employed as described in D. W. Grasse & K. A. Moxon, “Correcting the bias of spike field coherence estimators due to a finite number of spikes,” 104 J. Neurophysiol. 548-58 (2010). By breaking down the local field potential into its various frequency components and assessing how synchronized output-firing is to each of these signals, insight is gained into which frequency components are most influential to the observed changes in firing rate. By then identifying generators of this this oscillatory frequency, it can be inferred which regions of the brain may be motivating the changes in firing rate that are observed.

The device of the present invention can also be used to measure synchrony of neuronal activity. Synchrony can occur on varying temporal and spatial scales. At the cellular level, synchrony may occur between individual neurons as measured by cross-correlation or between individual neurons and the local field potential as measured by unit-field coherence (mentioned above). These measures occur within small volumes (<1 mm³) of tissue and when studying seizure recruitment within these volumes, these measures are made on a scale on the order of hundreds of milliseconds. The strength of correlation between the spike times of a neuron or neuron population and the phase of the concurrent local field potential at any given frequency may be measured as described more completely by Grasse and Moxon.

Measuring synchrony of neuron activity is important in understanding mechanisms of neuronal function, particularly in neurological disorders such as epilepsy. For example, the concept of synchronized neural activity is central to the present understanding of epilepsy. In focal epilepsy, seizures often start in a distinct focus and propagate to numerous downstream regions, so restricting an analysis to one region (such as the MTL) only provides a local view into changes associated with recruitment into seizure. To achieve a more comprehensive look at how seizures are spreading, it is necessary to look across brain structures, which changes the type of synchrony that must be analyzed. When looking across larger distances, i.e. between different brain regions, synchrony is generally discussed between the local field potentials at each region, and is usually measured in terms of field-field coherence or directed field-field coherence. As expected, the time scale of field-field coherence is correspondingly larger (on the order of seconds) because propagation lasts longer over greater distances. Importantly, whereas unit-field coherence provides a rough measure of which frequencies of the LFP most influence the firing of a neuronal population, directed field-field coherence is reflective of which frequencies are being used to convey information between brain structures. Directed field-field coherence may be calculated using established methods described in Franaszczuk et al., Electroencephalogr. Clin. Neurophysiol. 91:413-27 (1994); and Kaminski et al., Biol. Cybern. 210, 203-210 (1991)).

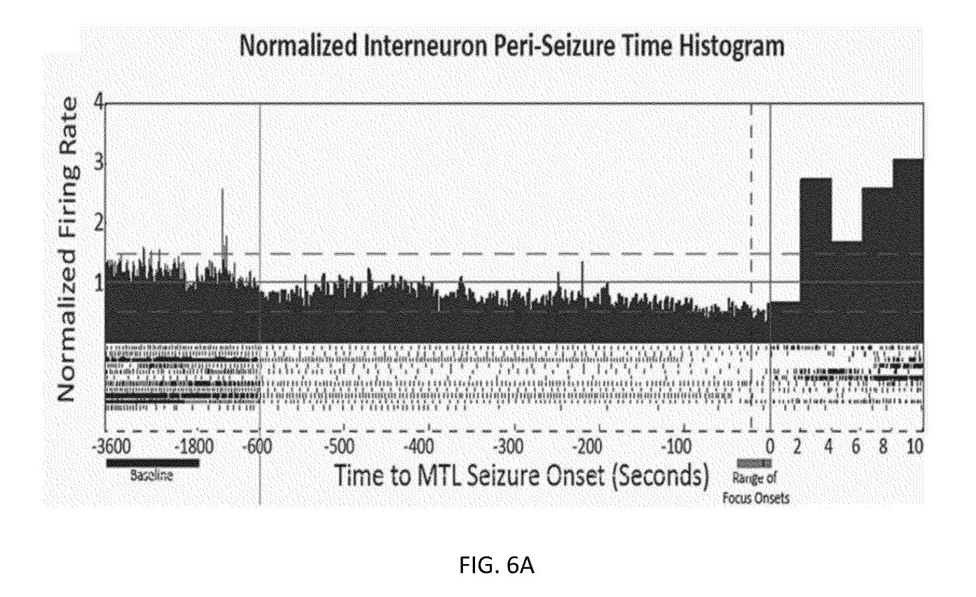

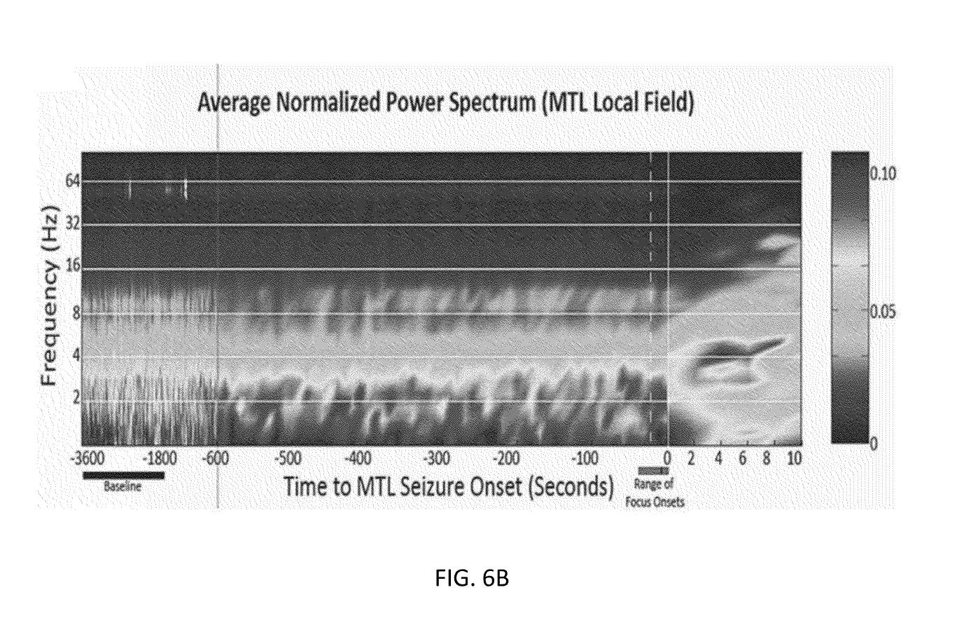

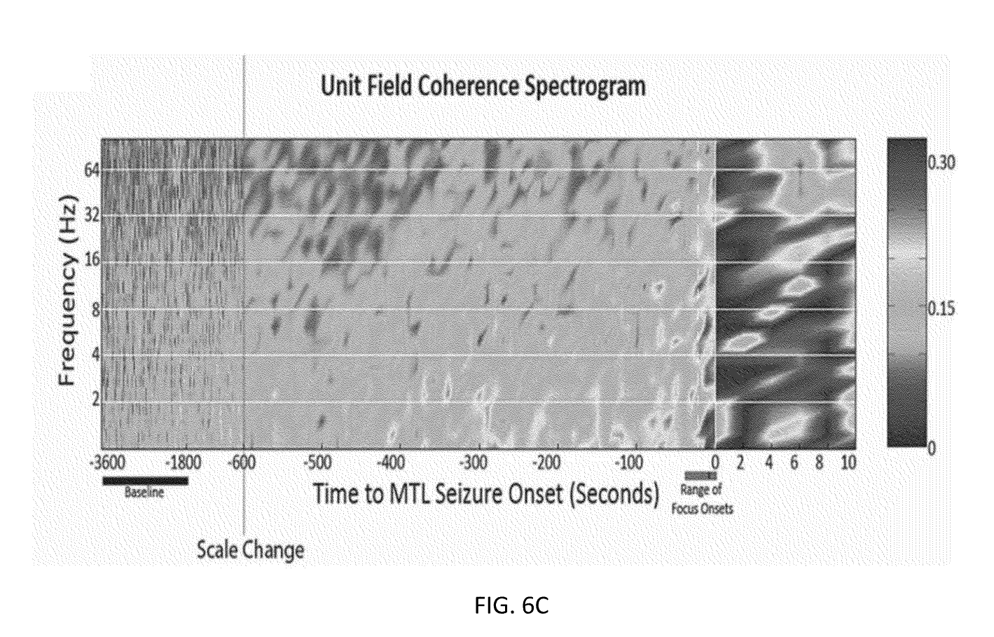

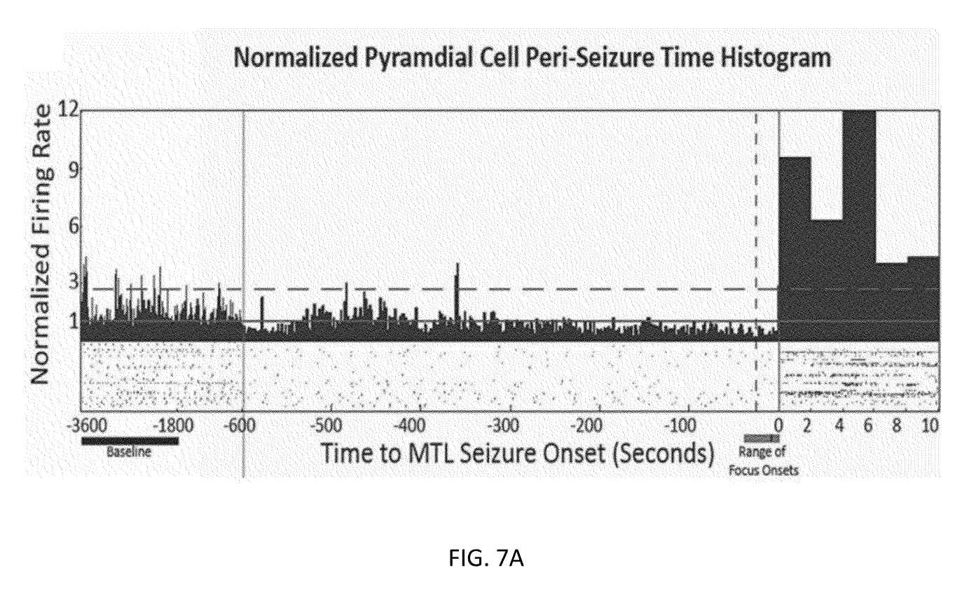

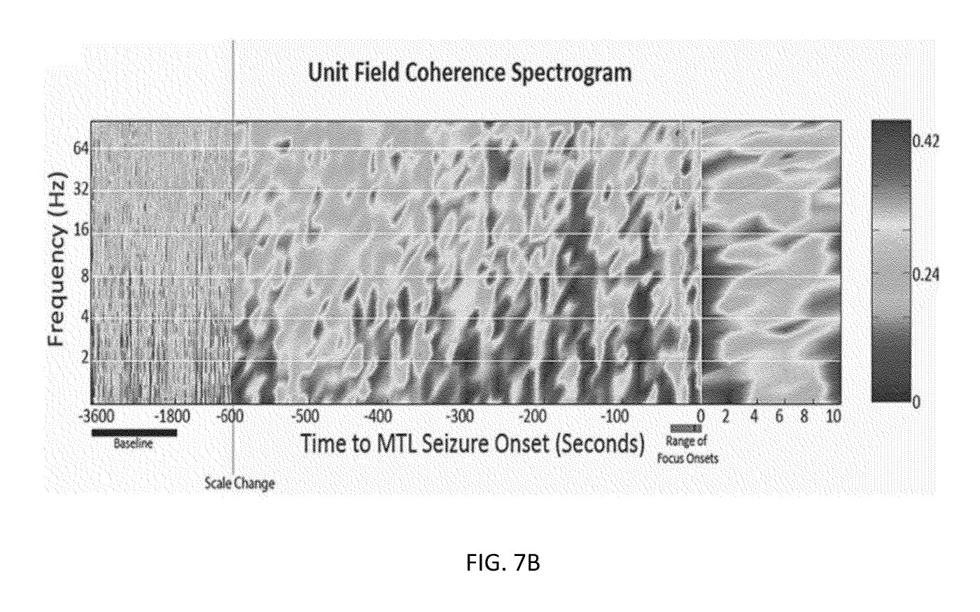

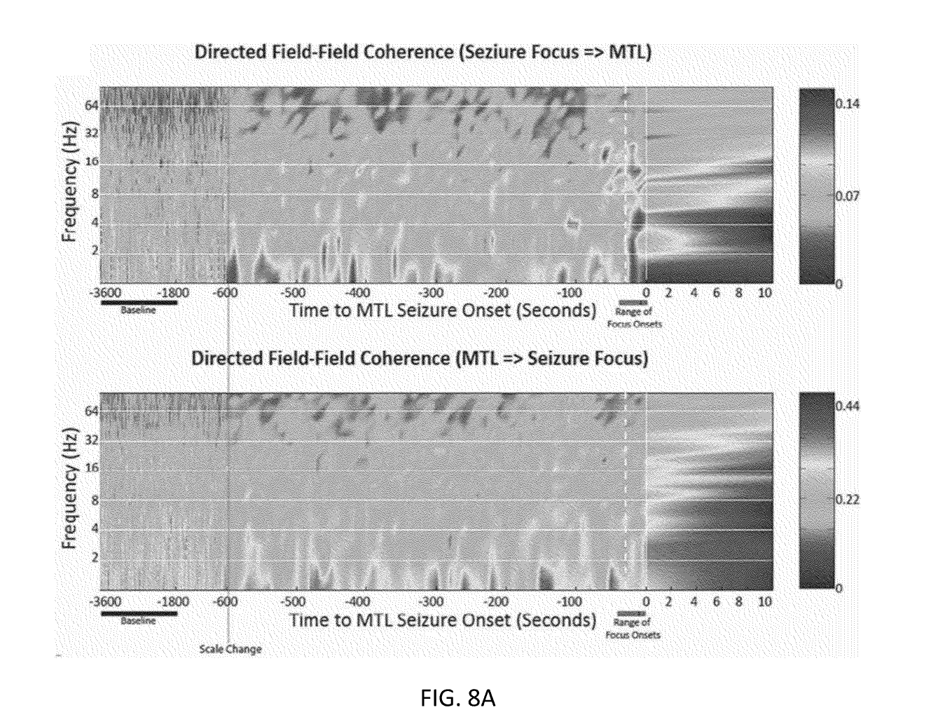

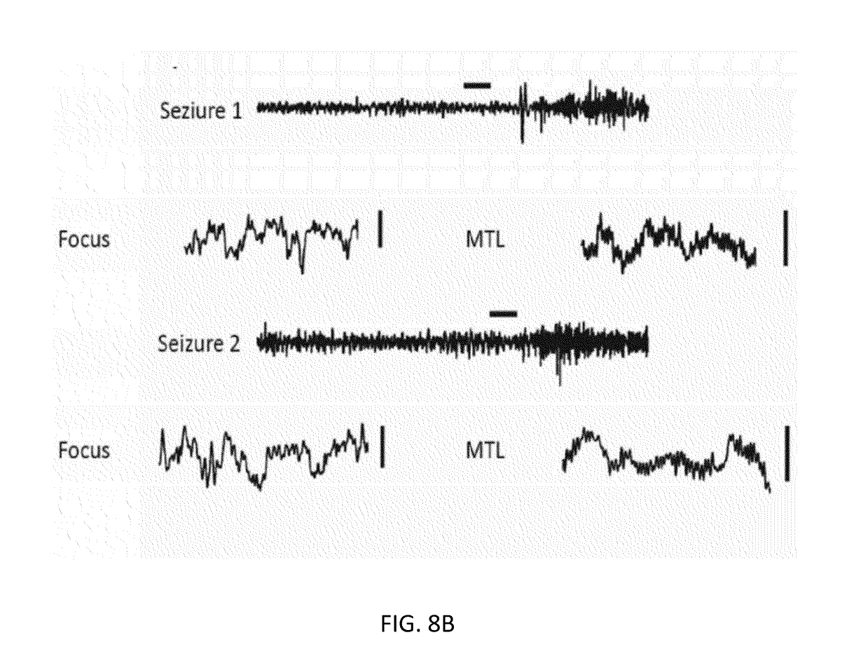

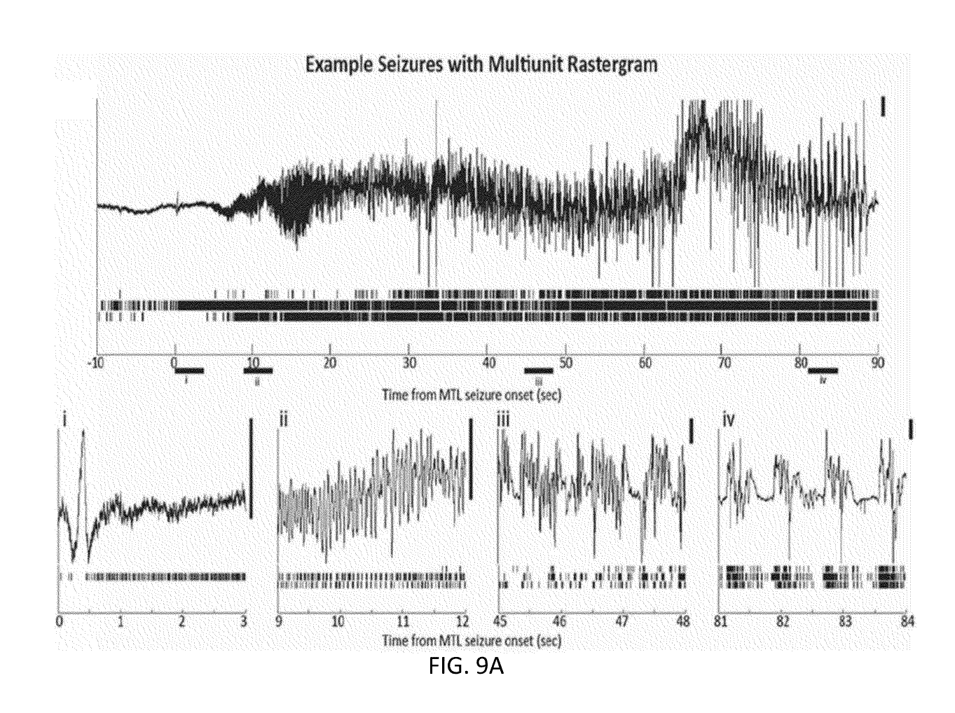

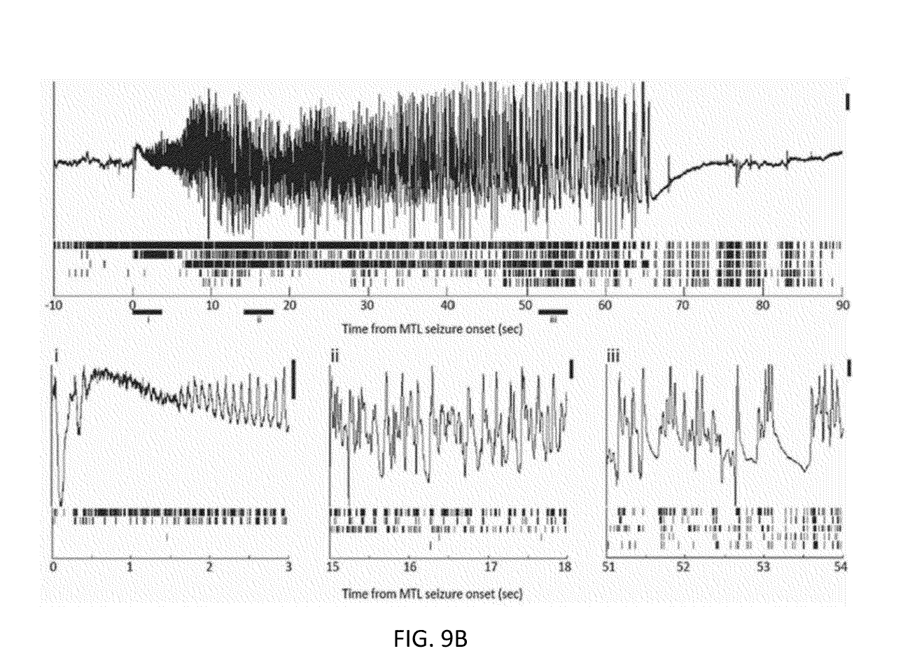

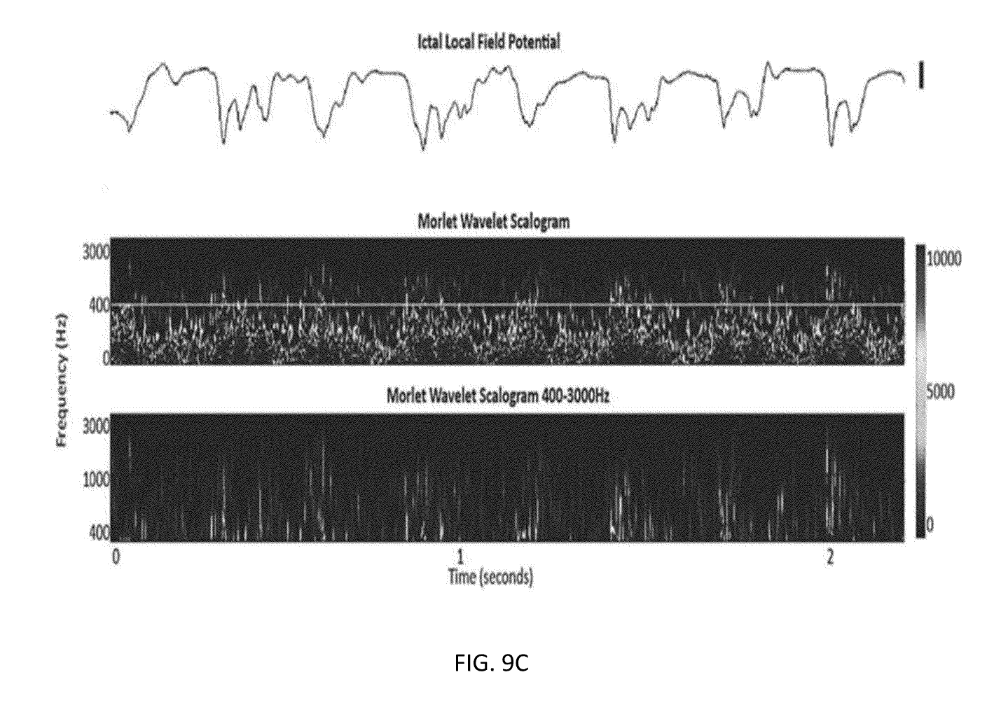

FIGS. 6A-6C, 7A-7B, 8A-8B, and 9A-9C show exemplary measurements of neuron firing rates, local field potential, and unit-field coherence and directed field-field coherence between various brain structures or regions in patients. FIGS. 6A and 7A, for example, show firing rates of interneurons and pyramidal cells respectively, during periods before and after onset of a seizure event. FIGS. 6B, 6C, and 7B show measurements of local field potential and unit-field coherence in the MTL. FIG. 8A shows measurements of directed field-field coherence between a seizure focus and the MTL. The measurements shown in FIGS. 6A-6C, 7A-7B, 8A-8B, and 9A-9C were obtained from recordings of neuron activity from chronically implanted electrodes in patients before and during onset of a seizure event and are further described in U.S. patent application Ser. No. 14/735,076.

As discussed herein, the device of the present invention is designed to overcome problems associated with recording from large populations of single neurons for long periods of time, such as the electrode itself failing or the immunological response in the tissue surrounding the microelectrode producing a glial scar and preventing single-neuron recording. Because the device is suited for long-term chronic single neuron recordings, the device and method of the present invention provide the ability to measure neuronal activity across wide spatial and temporal scales.

### EQUIVALENTS

Those skilled in the art will recognize, or be able to ascertain using no more than routine experimentation, many equivalents of the specific embodiments of the invention described herein. Such equivalents are intended to be encompassed by the following claims.

### INCORPORATION BY REFERENCE

The entire contents of all patents, published patent applications, and other references cited herein are hereby expressly incorporated herein in their entireties by reference.

1. An optically powered, integrated wireless neural electrode-telemetry module comprising:
a. a porous silicon wafer;
b. at least one neural electrode;
c. a low noise pre-amplifier;
d. an optical power converter;
e. a signal processor; and
f. a radio-frequency (RF) transmitter microchip.

2. The neural electrode-telemetry module of claim 1, wherein said optical power converter is adapted and configured to receive power from red or near-infrared illumination transmitted through a skull.

3. The neural electrode-telemetry module of claim 1, wherein said optical power converter is adapted and configured to receive power from optical signals carried by a fiber optic cable, wherein said fiber optic cable terminates near said optical power converter without physically contacting said optical power converter.

4. The neural electrode-telemetry module of claim 1, wherein said porous silicon wafer comprises at least one pharmacological agent.

5. The neural electrode-telemetry module of claim 4, wherein said pharmacological agent is selected from the group consisting: of brain derived neural growth factors (BDNF), nerve growth factor (NGF), Poloxamer 188, a cell adhesion molecule, glial filament associated proteins (GFAP), intermediate filament associated proteins (IFAP), endothelin-1, cytokinen, IL-6, and any combination thereof.

6. A method of chronically recording electrical activity from a single neuron in vivo, said method comprising the steps of:
a. implanting an optically powered, integrated wireless neural electrode-telemetry module into the brain or spinal cord of a subject, wherein said module comprises:
a porous silicon wafer ;
at least one neural electrode;
a low noise pre-amplifier;
an optical power converter;
a signal processor; and
a radio-frequency (RF) transmitter microchip;
b. providing an optical signal to power said optical power converter; and
c. receiving modulated radio signals from the RF transmitter microchip, wherein said modulated radio signals correspond to electrical activity of a single neuron.

7. The method of claim 6, wherein said optical signal comprises red or near-infrared illumination transmitted through a skull.

8. The method of claim 6, wherein said optical signal is carried by a fiber optic cable, wherein said fiber optic cable terminates near said optical power converter without physically contacting said optical power converter.

9. The method of claim 6, wherein said porous silicon wafer comprises at least one pharmacological agent.

10. The method of claim 9, wherein said pharmacological agent is selected from the group consisting of brain derived neural growth factors (BDNF), nerve growth factor (NGF), Poloxamer 188, a cell adhesion molecule, glial filament associated proteins (GFAP), intermediate filament associated proteins (IFAP), endothelin-1, cytokinen, IL-6, and any combination thereof.

11. The method of claim 6, wherein said subject is an animal.

12. The method of claim 11, wherein said animal is a mammal.

13. The method of claim 12, wherein said mammal is a human.
